(19) United States
(12) Patent Application Publication
(10) Pub. No.: US 2002/0048577 A1
(43) Pub. Date: Apr. 25, 2002

(54) Methods and devices to modulate the wound response

(75) Inventors: Paul Bornstein, Seattle, WA (US); Themis Kyriakides, Seattle, WA (US); Buddy Ratner, Seattle, WA (US); Cecilia Giachelli, Mill Creek, WA (US); Laura Martinson, San Diego, CA (US); Marta Scatena, Seattle, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: University of Washington

(21) Appl. No.: 09/919,770

(22) Filed: Jul. 31, 2001

Related U.S. Application Data
(60) Provisional application No. 60/222,071, filed on Aug. 1, 2000.

(51) Int. Cl. A61K 48/00; A61K 38/48

(52) U.S. Cl. 424/94.63; 424/422; 514/44

(57) ABSTRACT

In one aspect, the invention provides methods of modulating the amount and/or biological activity of thrombospondin 2 or osteopontin in an animal. The methods comprise the step of introducing into the animal an amount of osteopontin, and/or a thrombospondin 2 antagonist, effective to modulate the amount or biological activity of thrombospondin 2 or osteopontin in the animal. In another aspect, the invention provides medical devices comprising (a) a device body; and (b) a surface layer attached to the device body, the surface layer including an amount of an agonist or antagonist of a matricellular protein sufficient to reduce the foreign body response against the medical device, wherein the medical device is adapted to be affixed to, or implanted within, the soft tissue of an animal.

### CROSS-REFERENCE(S) TO RELATED APPLICATION(S)

The present application claims the benefit of U.S. provisional patent application serial No. 60/222,071, filed Aug. 1, 2000, under 35 U.S.C. §119.

### GOVERNMENT RIGHTS

The present invention was funded, at least in part, by National Science Foundation grant number ECC 9529161, and by National Institutes of Health grant number AR 45418. The United States government has certain rights in the invention.

### FIELD OF THE INVENTION

The present invention relates to methods for modulating the wound response, such as improving the wound response, or reducing the foreign body response against a medical device implanted into an animal body.

### BACKGROUND OF THE INVENTION

Animals exhibit a variety of physiological and biochemical responses at the site of tissue damage or injury. These physiological and biochemical responses are collectively referred to as the wound response. The wound response facilitates the repair or replacement of the damaged or destroyed tissue. In some situations, however, wounded tissue exhibits a chronic wound response that adversely affects the health or well-being of the wounded animal.

The implantation of a medical device into soft tissue elicits a wound response. This type of wound response is called the foreign body response and results in the encapsulation of the implant by a poorly-vascularized, collagenous, capsule that can compromise the function of the implant. In addition, the continued presence of the implant can lead to a chronic inflammatory response that is mediated, in part, by macrophages.

Thrombospondin-2 (TSP2) is a secreted, extracellular matrix glycoprotein with potent anti-angiogenic activity (Bornstein et al., 2000, Matrix Biology 19: 557-568). Osteopontin (OPN) is a secreted, phosphorylated glycoprotein that contains cell adhesion domains (Fisher et al., Genomics 7, 491-502 (1990)). The present inventors have discovered that modulation of the amount and/or biological activity of osteopontin (OPN) and/or thrombospondin 2 (TSP2) in an animal can be utilized to modulate the wound response, such as the foreign body response to an implanted medical device.

### SUMMARY OF THE INVENTION

In accordance with the foregoing, in one aspect the present invention provides methods of modulating the amount and/or biological activity of thrombospondin 2 or osteopontin in an animal, the methods comprising the step of introducing into the animal an amount of a molecule, selected from the group consisting of osteopontin and a thrombospondin 2 antagonist, effective to modulate the amount and/or biological activity of thrombospondin 2 or osteopontin in the animal. In this context, when used with reference to OPN, the term “modulating” means increasing or decreasing the amount and/or biological activity of OPN. In this context, when used with reference to TSP2, the term “modulating” means decreasing the amount and/or biological activity of TSP2. In some embodiments of this aspect of the invention, the amount and/or biological activity of OPN is increased. In some embodiments of this aspect of the invention, the amount and/or biological activity of TSP2 is decreased.

In another aspect, the present invention provides methods of improving the wound response in an animal, the methods comprising the step of introducing into the animal an amount of a molecule, selected from the group consisting of osteopontin and a thrombospondin 2 antagonist, effective to improve the wound response in the animal.

In another aspect, the present invention provides methods of reducing the foreign body response in an animal, the methods comprising the step of introducing into the animal an amount of a molecule, selected from the group consisting of osteopontin and a thrombospondin 2 antagonist, effective to reduce the foreign body response in the animal. Typically, in the practice of the methods of the invention to improve the wound response, and/or to reduce the foreign body response, the amount and/or biological activity of osteopontin is increased, and/or the amount and/or biological activity of thrombospondin-2 is decreased.

The methods of the invention can be used to modulate the wound response in any situation where modulation of the wound response is desirable, including situations in which it is desirable to reduce the foreign body response, and including situations in which it is desirable to improve the wound response.

In another aspect, the present invention provides medical devices, each medical device comprises (a) a device body; and (b) a surface layer attached to the device body, the surface layer including an amount of an agonist or antagonist of a matricellular protein sufficient to reduce the foreign body response against the medical device, wherein the device is adapted to be affixed to, or implanted within, the soft tissue of an animal. Thus, the medical devices of the invention are useful in any situation in which it is desired to reduce the foreign body response against an implanted medical device.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows a perspective view of a representative medical device of the invention with a portion of the surface layer removed to expose the underlying device body.

FIG. 2 shows a transverse cross-section of the medical device of FIG. 1.

FIG. 3 shows the porous matrix structure of the surface layer of the representative medical device shown in FIG. 1.

FIG. 4 shows a perspective view of a representative medical device of the invention that includes two surface layers disposed one upon the other. The outer surface layer includes osteopontin protein, and the inner surface layer includes an antagonist of thrombospondin 2.

FIG. 5 shows a perspective view of a representative medical device of the invention that includes a device body, and a surface layer disposed on the device body. The surface layer includes a first area, including a first agonist or first antagonist of a matricellular protein, and a second area, including a second agonist or second antagonist of a matricellular protein. The first agonist is different from the second agonist, and the first antagonist is different from the second antagonist.

FIG. 6 shows data showing the extent of vascularization of foreign body capsules formed around devices implanted into mice. The devices were each made from a millipore filter coated with a collagen matrix. The collagen matrices were impregnated with a plasmid including either a TSP2 sense (S), or TSP2 antisense (AS), nucleic acid molecule. P represents devices coated with a collagen matrix that was not impregnated with a plasmid. The devices were implanted into either TSP2-null (−/−) or normal control (+/+) mice. The x-axis shows the number of weeks (two or four) of implantation within a mouse. The y-axis shows the number of blood vessels, per visual field, within each foreign body capsule as viewed under a microscope. “Control” represents implanted millipore filters that were not coated with collagen.

FIG. 7 shows the number of foreign body giant cells produced at the site of implantation of fixed bovine pericardium samples into either OPN null mice (OPN knockout mice) or normal control mice. The results from seven OPN null mice and seven control mice were measured. The numbers of foreign body giant cells was measured at 14 days and 30 days post implantation.

FIG. 8A shows the foreign body capsule thickness for polyethylene discs (PE), polyethylene discs coated with tetraglyme (PE glyme), and polyethylene discs coated with tetraglyme to which are covalently attached osteopontin protein molecules (PE glyme OPN). The discs were implanted into mice and the foregoing parameters measured after four weeks.

FIG. 8B shows the macrophage score (a measure of the number of macrophages in each disc) for the discs described in the legend for FIG. 8A.

### DETAILED DESCRIPTION OF THE PREFERRED EMBODIMENT

Unless specifically defined herein, all terms used herein have the same meaning as they would to one skilled in the art of the present invention. The following definitions are provided in order to provide clarity with respect to the terms as they are used in the specification and claims to describe the present invention.

As used herein, the term “wound response” refers collectively to the biochemical and physiological repair processes elicited at the site of wounding in, or on, an animal body, such as by the implantation of a medical device. The wound response is characterized by a transient inflammatory reaction followed by an invasion of collagen secreting fibroblasts and new vasculature formation in the wound bed. These events are followed by granulation tissue formation and remodeling.

The term “matricellular protein” refers to proteins that have the ability to simultaneously interact with a component of the extracellular matrix and a component of the cell surface. Some matricellular proteins can interact with growth factors and/or proteinases. Matricellular proteins function primarily to regulate cell adhesion, movement, and function. Examples of matricellular proteins include TSP1 (Chen et al., Matrix Biology 19: 597-614), TSP2 (Bornstein et al., Matrix Biology 19: 557-568) OPN (Giachelli and Steitz, Matrix Biology 19: 615-622) tenascin-C (Jones and Jones, Matrix Biology 19: 581-596)and SPARC (Brekken and Sage, Matrix Biology 19: 569-580). Each of the foregoing publications are incorporated herein by reference.

The term “foreign body response” refers to a type of wound response in which a poorly-vascularized, collagenous, capsule forms around a structure (such as a medical device) implanted into an animal body.

The phrase “soft tissue of an animal” refers to any animal tissue except bone, nail, or hair. The phrase “soft tissue of an animal” includes, for example, muscle and skin.

The term “hybridize under stringent conditions”, and grammatical equivalents thereof, refers to the ability of a nucleic acid molecule to hybridize to a target nucleic acid molecule (such as a target nucleic acid molecule immobilized on a DNA or RNA blot, such as a Southern blot or Northern blot) under defined conditions of temperature and salt concentration. Typically, stringent hybridization conditions are no more than 25° C. to 30° C. (for example, 10° C.) below the melting temperature (Tm) of the native duplex. By way of non-limiting example, representative salt and temperature conditions for achieving stringent hybridization are: 5× SSC, at 65° C., or equivalent conditions; see generally, Sambrook et al. Molecular Cloning: A Laboratory Manual, 2nd ed., Cold Spring Harbor Press, 1987; Ausubel et al., Current Protocols in Molecular Biology, Greene Publishing, 1987. Tm for nucleic acid molecules greater than about 100 bases can be calculated by the formula Tm=81.5+0.41%(G+C)−log (Na⁺). For oligonucleotide molecules less than 100 bases in length, exemplary hybridization conditions are 5 to 10° C. below Tm. On average, the Tm of a short oligonucleotide duplex is reduced by approximately (500/oligonucleotide length)° C.

The abbreviation “SSC” refers to a buffer used in nucleic acid hybridization solutions. One liter of the 20× (twenty times concentrate) stock SSC buffer solution (pH 7.0) contains 175.3 g sodium chloride and 88.2 g sodium citrate.

The term “sequence identity” or “percent identical” as applied to nucleic acid molecules is the percentage of nucleic acid residues in a candidate nucleic acid molecule sequence that are identical with a subject nucleic acid molecule sequence (such as the nucleic acid molecule sequence set forth in SEQ ID NO: 1), after aligning the sequences to achieve the maximum percent identity, and not considering any nucleic acid residue substitutions as part of the sequence identity. No gaps are introduced into the candidate nucleic acid sequence in order to achieve the best alignment.

Nucleic acid sequence identity can be determined in the following manner. The subject polynucleotide molecule sequence is used to search a nucleic acid sequence database, such as the Genbank database (accessible at Website http://www.ncbi.nlm.nih.gov/blast/), using the program BLASTN version 2.1 (based on Altschul et al., Nucleic Acids Research 25: 3389-3402 (1997)). The program is used in the ungapped mode. Default filtering is used to remove sequence homologies due to regions of low complexity as defined in Wootton, J. C. and S. Federhen, Methods in Enzymology 266: 554-571 (1996). The default parameters of BLASTN are utilized.

The term “sequence identity” or “percent identical” as applied to protein molecules is the percentage of amino acid residues in a candidate protein molecule sequence that are identical with a subject protein sequence (such as the protein sequence set forth in SEQ ID NO:2), after aligning the sequences to achieve the maximum percent identity. No gaps are introduced into the candidate protein sequence in order to achieve the best alignment.

Amino acid sequence identity can be determined in the following manner. The subject protein sequence is used to search a protein sequence database, such as the GenBank database (accessible at web site http://www.ncbi.nln.nih.gov/blast/), using the BLASTP program. The program is used in the ungapped mode. Default filtering is used to remove sequence homologies due to regions of low complexity. The default parameters of BLASTP are utilized. Filtering for sequences of low complexity utilize the SEG program.

The term “antibody” encompasses polyclonal and monoclonal antibody preparations, CDR-grafted antibody preparations, as well as preparations including hybrid antibodies, altered antibodies, F(AB)′₂ fragments, F(AB) molecules, Fv fragments, single domain antibodies, chimeric antibodies and functional fragments thereof which exhibit immunological binding properties of the parent antibody molecule. The antibodies can also be humanized.

In one aspect, the present invention provides methods of modulating the amount and/or biological activity of thrombospondin 2 (TSP2) and/or osteopontin (OPN) in an animal. The methods of this aspect of the invention comprise the step of introducing into the animal an amount of a molecule, selected from the group consisting of OPN and a TSP2 antagonist, effective to modulate the amount and/or biological activity of TSP2 or OPN in the animal. In this context, when used with reference to OPN, the term “modulating” means increasing or decreasing the amount and/or biological activity of OPN. In this context, when used with reference to TSP2, the term “modulating” means decreasing the amount and/or biological activity of TSP2. In some embodiments of this aspect of the invention, the amount and/or biological activity of OPN is increased. In some embodiments of this aspect of the invention, the amount and/or biological activity of TSP2 is decreased. The methods described in the present invention are applicable to any animal, including mammals, such as human beings. The methods of this aspect of the invention can be used to modulate the wound response in any situation where modulation of the wound response is desirable, including situations in which it is desirable to reduce the foreign body response and including situations in which it is desirable to improve the wound response (e.g. improve the rate of wound healing at the site of a cut, abrasion, or burn to soft tissue). In some embodiments, in order to modulate the wound response, the amount and/or biological activity of OPN is increased, and/or the amount and/or biological activity of TSP2 is decreased.

The methods of this aspect of the invention can be used, for example, to improve the wound response (e.g., by increasing the magnitude of one or more of the biochemical and/or physiological and/or physical responses that make up the wound response, and/or reducing the duration of the wound response), such as at the site of a cut, abrasion or burn (e.g. by applying to the cut, abrasion or burn, an article, such as an adhesive strip, that includes an amount of OPN and/or a TSP2 antagonist, that is effective to improve the wound response). Improved wound response is especially important, for example, in a diabetic person, where cuts, abrasions and burns are slow to heal.

The methods of this aspect of the invention can also be used, for example, to reduce the foreign body response, (e.g. by reducing the magnitude of one or more of the biochemical and/or physiological and/or physical responses that make up the foreign body response, and/or reducing the duration of the foreign body response). Representative examples of situations in which it is desirable to reduce the foreign body response include the reduction of the foreign body response against an implanted medical device, thereby prolonging the working lifetime of the implanted device.

Other examples of situations where it is desirable to use the methods of the invention to reduce the foreign body response include: reduction of the foreign body response at the site of an implanted vascular stent, thereby preventing or delaying restenosis at the location of the stent; and reduction of the foreign body response elicited by tissues or organs implanted into an animal body, thereby promoting the acceptance of the implanted tissue or organ by the host body.

A reduction in the foreign body response is characterized by at least one of the following changes in a component of the foreign body response that occurs as a result of treatment of animal tissue in accordance with the methods of the invention: a decrease in the amount of fibrosis (measured, for example, by a decrease in hydroxy-proline content which indicates the level of collagen in the foreign body capsule); a decrease in the amount of inflammation (measured, for example, by counting the number of inflammatory cells, and the number of foreign body giant cells, in histological sections; or measuring the levels of cytokines, such as interleukin and monocyte chemoattractant protein, in wound extracts by ELISA); an increase in the amount of vascularization of the capsule formed as part of the foreign body response (measured, for example, by visualizing blood vessels in histological sections with anti-PECAM1 antibody and the peroxidase reaction; the number of vessels and their average size are estimated with imaging software such as Metamorph); an increase in the amount of permeability of the capsule formed as part of the foreign body response (measured, for example, as the release of traceable chemicals from implanted devices, or ability of implanted sensors to sense plasma levels of molecules such as glucose); a decrease in the amount of the capsule formed around the foreign body (capsule thickness can be measured from histological sections with the aid of ocular micrometers); and a decrease in the amount of contraction of collagen fibers within the capsule that is formed as part of the foreign body response (measured as tensile strength of the capsule or induced shape change on malleable implants). The decrease, or increase, of any of the foregoing parameters can be a decrease, or increase, relative to the amount of the parameter present before treatment in accordance with the methods of the invention; or a decrease, or increase, relative to the amount of the parameter present in control tissue that is not treated in accordance with the methods of the present invention.

Thus, in one aspect, the present invention provides methods of improving the wound response in an animal, the methods comprising the step of introducing into the animal an amount of a molecule, selected from the group consisting of osteopontin and a thrombospondin 2 antagonist, effective to improve the wound response in the animal. In another aspect, the present invention provides methods of reducing the foreign body response in an animal, the methods comprising the step of introducing into the animal an amount of a molecule, selected from the group consisting of osteopontin and a thrombospondin 2 antagonist, effective to reduce the foreign body response in the animal. Typically, in the practice of the methods of the invention to improve the wound response, and/or to reduce the foreign body response, the amount and/or biological activity of osteopontin is increased, and/or the amount and/or biological activity of thrombospondin-2 is decreased.

Any OPN protein that improves the wound response and/or reduces the foreign body response is useful in the practice of the present invention. OPN proteins useful in the methods of the present invention include naturally purified OPN protein (which may be chemically modified after purification), chemically synthesized OPN protein, and OPN protein produced by recombinant techniques from a prokaryotic or eukaryotic host, including, for example, bacterial, yeast, insect, mammalian, avian and higher plant cells.

OPN fragments that improve the wound response and/or reduce the foreign body response are also useful in the practice of the present invention. Also, modified OPN proteins, or fragments thereof, that improve the wound response and/or reduce the foreign body response are useful in the practice of the present invention. Modifications can include those that are introduced during or after translation, (e.g., by glycosylation, proteolytic cleavage, linkage to an antibody molecule or other cellular ligand). Modifications also include N-terminal modifications, which result from expression in a particular recombinant host, such as, for example, N-terminal methylation which occurs in certain bacterial (e.g. E. coli) expression systems. Modifications also include mutants in which amino acid substitutions are made.

OPN protein, or OPN fragments, can be recovered and purified by any applicable purification method, including ammonium sulfate or ethanol precipitation, acid extraction, anion or cation exchange chromatography, gel filtration, hydrophobic interaction chromatography, affinity chromatography, hydroxylapatite chromatography, and high performance liquid chromatography (“HPLC”).

The cDNA molecule set forth in SEQ ID NO:1 encodes a representative example of OPN (consisting of the amino acid sequence set forth in SEQ ID NO:2) useful in the practice of the invention. Other representative examples of useful OPN proteins include OPN proteins that are at least 70% identical to the OPN protein set forth in SEQ ID NO.2.

The amount and/or biological activity of OPN in an animal can be modulated (for example increased) by any suitable method, such as one or more of the following, representative, methods: the delivery of nucleic acid molecules encoding OPN into the body of an animal; increasing the level of endogenous OPN transcription and/or translation within the body of an animal; delivery of OPN protein (or OPN fragments that retain the ability to modulate the wound response) into the body of an animal by implanting into the body of an animal, or attaching to the body of an animal, a structure comprising OPN, or OPN peptides retaining the ability to modulate the wound response, disposed on a surface of the structure that contacts tissue of the animal body when the structure is implanted therein.

OPN protein, or OPN peptides retaining the ability to modulate the wound response, can be delivered into the body of an animal by any suitable means. By way of representative example, OPN protein, or fragments thereof, can be introduced into an animal body by application to a bodily membrane capable of absorbing the protein, for example the nasal, gastrointestinal and rectal membranes. The protein is typically applied to the absorptive membrane in conjunction with a permeation enhancer. (See, e.g., V. H. L. Lee, Crit. Rev. Ther. Drug Carrier Syst., 5:69 (1988); V. H. L. Lee, J. Controlled Release, 13:213 (1990); V. H. L. Lee, Ed., Peptide and Protein Drug Delivery, Marcel Dekker, New York (1991); A. G. DeBoer et al., J. Controlled Release, 13:241 (1990)). For example, STDHF is a synthetic derivative of fusidic acid, a steroidal surfactant that is similar in structure to the bile salts, and has been used as a permeation enhancer for nasal delivery. (W. A. Lee, Biopharm. Nov./Dec., 22, 1990).

The OPN protein, or fragments thereof, may be introduced in association with another molecule, such as a lipid, to protect the protein from enzymatic degradation. For example, the covalent attachment of polymers, especially polyethylene glycol (PEG), has been used to protect certain proteins from enzymatic hydrolysis in the body and thus prolong half-life (F. Fuertges, et al., J. Controlled Release, 11:139 (1990)). Many polymer systems have been reported for protein delivery (Y. H. Bae, et al., J. Controlled Release, 9:271 (1989); R. Hori, et al., Pharm. Res., 6:813 (1989); I. Yamakawa, et al., J. Pharm. Sci., 79:505 (1990); I. Yoshihiro, et al., J. Controlled Release, 10:195 (1989); M. Asano, et al., J. Controlled Release, 9:111 (1989); J. Rosenblatt et al., J. Controlled Release, 9:195 (1989); K. Makino, J. Controlled Release, 12:235 (1990); Y. Takakura et al., J. Pharm. Sci., 78:117 (1989); Y. Takakura et al., J. Pharm. Sci., 78:219 (1989)).

For transdermal applications, the OPN protein, or fragments thereof, may be combined with other suitable ingredients, such as carriers and/or adjuvants. There are no limitations on the nature of such other ingredients, except that they must be pharmaceutically acceptable and efficacious for their intended administration, and cannot degrade the activity of the active ingredients of the composition. Examples of suitable vehicles include ointments, creams, gels, or suspensions, with or without purified collagen. The OPN protein, or fragments thereof, also may be impregnated into transdermal patches, plasters, and bandages, preferably in liquid or semi-liquid form.

The amount and/or biological activity of OPN in an animal can be increased, for example, by delivery of nucleic acid molecules encoding OPN, or a biologically active fragment thereof, into the body of an animal. By way of example, a vector which includes a nucleic acid molecule (typically a DNA molecule) that encodes an OPN protein can be introduced into any suitable host cell, including animal and human cells, and the encoded OPN protein expressed therein. The vector can be introduced into host cells in vitro, and the modified cells introduced into the body of an animal, or the vector can be introduced into cells, in vivo, within the body of an animal. Any art-recognized gene delivery method can be used to introduce a vector into one or more cells for expression therein, including: transduction, transfection, transformation, direct injection, electroporation, virus-mediated gene delivery, amino acid-mediated gene delivery, biolistic gene delivery, lipofection and heat shock. See, generally, Sambrook et al, supra. Representative, non-viral, methods of gene delivery into cells are disclosed in Huang, L., Hung, M-C, and Wagner, E., Non-Viral Vectors for Gene Therapy, Academic Press, San Diego, Calif. (1999).

Expression vectors useful for expressing OPN protein, or biologically active fragments thereof, include chromosomal, episomal, and virus-derived vectors, e.g., vectors derived from bacterial plasmids, bacteriophages, yeast episomes, yeast chromosomal elements, viruses such as baculoviruses, papova viruses, vaccinia viruses, adenoviruses, fowl pox viruses, pseudorabies viruses and retroviruses, and vectors derived from combinations thereof, such as cosmids and phagemids. In certain embodiments in this regard, the vectors provide for specific expression, which may be inducible and/or cell type-specific. Among such expression vectors are those inducible by environmental factors that are easy to manipulate, such as temperature and nutrient additives.

For example, a coding sequence for OPN, or a biologically active fragment thereof, can be introduced into cells in situ, or after removal of the cells from the body, by means of viral vectors. For example, retroviruses are RNA viruses that have the ability to insert their genes into host cell chromosomes after infection. Retroviral vectors have been developed that lack the genes encoding viral proteins, but retain the ability to infect cells and insert their genes into the chromosomes of the target cell (A. D. Miller, Hum. Gen. Ther. 1:5-14 (1990)). Adenoviral vectors are designed to be administered directly to patients. Unlike retroviral vectors, adenoviral vectors do not integrate into the chromosome of the host cell. Instead, genes introduced into cells using adenoviral vectors are maintained in the nucleus as an extrachromosomal element (episome) that persists for a limited time period. Adenoviral vectors will infect dividing and non-dividing cells in many different tissues in vivo including airway epithelial cells, endothelial cells, hepatocytes and various tumors (B. C. Trapnell, Adv Drug Del Rev. 12:185-199 (1993)).

Another viral vector is the herpes simplex virus; a large, double-stranded DNA virus. Recombinant forms of the vaccinia virus can accommodate large inserts and are generated by homologous recombination. To date, this vector has been used to deliver, for example, interleukins (ILs), such as human IL-1β and the costimulatory molecules B7-1 and B7-2 (G. R. Peplinski et al., Ann. Surg. Oncol. 2:151-9 (1995); J. W. Hodge et al., Cancer Res. 54:5552-55 (1994)).

A plasmid vector can be introduced into mammalian cells in a precipitate, such as a calcium phosphate precipitate, or in a complex with a charged lipid (e.g., LIPOFECTAMINE™; Life Technologies, Inc.; Rockville, Md.) or in a complex with a virus (such as an adenovirus) or components of a virus (such as viral capsid peptides). If the vector is a virus, it may be packaged in vitro using an appropriate packaging cell line and then transduced into host cells.

For example, a vector may be formulated for delivery either encapsulated in a lipid particle, a liposome, a vesicle, or a gene activated collagen matrix. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers.

Recently, liposomes were developed with improved serum stability and circulation half-times (see, e.g., U.S. Pat. No. 5,741,516). Furthermore, various methods of liposome and liposome-like preparations as potential drug carriers have been reviewed (see, e.g., U.S. Pat. Nos. 5,567,434; 5,552,157; 5,565,213; 5,738,868 and 5,795,587).

Additionally, studies have demonstrated that intramuscular injection of plasmid DNA formulated with 5% PVP (50,000 kDa) increases the level of reporter gene expression in muscle as much as 200-fold over the levels found with injection of DNA in saline alone (R. J. Mumper et al., Pharm. Res. 13:701-709 (1996); R. J. Mumper et al., Proc. Intern. Symp. Cont. Rol. Bioac. Mater. 22:325-326 (1995)). Intramuscular administration of plasmid DNA results in gene expression that lasts for many months (J. A. Wolff et al., Hum. Mol. Genet. 1:363-369 (1992); M. Manthorpe et al., Hum. Gene Ther. 4:419-431 (1993); G. Ascadi et al., New Biol. 3:71-81 (1991), D. Gal et al., Lab. Invest. 68:18-25 (1993)).

Various devices have been developed for enhancing the availability of DNA to a target cell. A simple approach is to contact the target cell physically with catheters or implantable materials containing DNA (G. D. Chapman et al., Circulation Res. 71:27-33 (1992)). Another method for achieving gene transfer involves using a fibrous collagen implant material soaked in a solution of DNA shortly before being placed in the site in which one desires to achieve gene transfer. The matrix may become impregnated with a gene DNA segment simply by soaking the matrix in a solution containing the DNA, such as a plasmid solution.

Another approach is to utilize needle-free, jet injection devices which project a column of liquid directly into the target tissue under high pressure. (P. A. Furth et al., Anal. Biochem. 20:365-368 (1992); (H. L. Vahlsing et al., J. Immunol. Meth. 175:11-22 (1994); (F. D. Ledley et al., Cell Biochem. 18A:226 (1994)).

Another device for gene delivery is the “gene gun” or Biolistic™, a ballistic device that projects DNA-coated micro-particles directly into the nucleus of cells in vivo. Once within the nucleus, the DNA dissolves from the gold or tungsten microparticle and can be expressed by the target cell. This method has been used effectively to transfer genes directly into the skin, liver and muscle (N. S. Yang et al., Proc. Natl. Acad. Sci. 87:9568-9572 (1990); L. Cheng et al., Proc. Natl. Acad. Sci. USA. 90:4455-4459 (1993); R. S. Williams et al., Proc. Natl. Acad. Sci. 88:2726-2730 (1991)).

OPN proteins, or fragments thereof, may be immobilized onto (or within) a surface of an implantable or attachable medical device. The modified surface will typically be in contact with living tissue after implantation into an animal body. By “implantable or attachable medical device” is intended any device that is implanted into, or attached to, tissue of an animal body, during the normal operation of the device (e.g., implantable drug delivery devices). Such implantable or attachable medical devices can be made from, for example, nitrocellulose, diazocellulose, glass, polystyrene, polyvinylchloride, polypropylene, polyethylene, dextran, Sepharose, agar, starch, and nylon. Linkage of the protein to a device can be accomplished by any technique that does not destroy the biological activity of the linked protein, for example by attaching one or both ends of the protein to the device. Attachment may also be made at one or more internal sites in the protein. Multiple attachments (both internal and at the ends of the protein) may also be used. A surface of an implantable or attachable medical device can be modified to include functional groups (e.g., carboxyl, amide, amino, ether, hydroxyl, cyano, nitrido, sulfanamido, acetylinic, epoxide, silanic, anhydric, succinimic, azido) for protein immobilization thereto. Coupling chemistries include, but are not limited to, the formation of esters, ethers, amides, azido and sulfanamido derivatives, cyanate and other linkages to the functional groups available on OPN proteins or fragments. OPN protein, or fragments thereof, can also be attached non-covalently by the addition of an affinity tag sequence to the protein, such as GST (Smith, D. B., and Johnson, K. S., Gene 67:31 (1988)), polyhistidines (Hochuli, E., et al., J. Chromatog. 411:77 (1987)), or biotin. Such affinity tags may be used for the reversible attachment of the protein to a device. The medical devices of the invention described herein can be used to deliver OPN proteins, or fragments thereof, to an animal body.

Methods of delivery of OPN proteins, or fragments thereof, also include administration by oral, pulmonary, parenteral (e.g., intramuscular, intraperitoneal, intravenous (IV) or subcutaneous injection), inhalation (such as via a fine powder formulation), transdermal, nasal, vaginal, rectal, or sublingual routes of administration, and can be formulated in dosage forms appropriate for each route of administration.

In another embodiment of the methods of the present invention, the amount and/or biological activity of thrombospondin 2 (TSP2) is decreased in an animal by a method comprising the step of introducing into the animal an amount of a TSP2 antagonist effective to decrease the amount and/or biological activity of TSP2 in the animal. In the practice of this aspect of the invention, representative TSP2 antagonists include: TSP2 antisense nucleic acid molecules (such as antisense mRNA, antisense DNA or antisense oligonucleotides), TSP2 ribozymes, and molecules that inhibit the biological activity of TSP2 (such as anti-TSP2 antibodies, or a blocking peptide which interacts with TSP2 or a TSP2 receptor), thereby preventing TSP2 from eliciting a biological response. The methods of this aspect of the invention can be used to improve the wound response in an animal, and/or reduce the foreign body response in an animal.

An antisense nucleic acid molecule may be constructed in a number of different ways provided that it is capable of interfering with the expression of a target gene. For example, an antisense nucleic acid molecule can be constructed by inverting the coding region (or a portion thereof) of TSP2 relative to its normal orientation for transcription to allow the transcription of its complement.

The antisense nucleic acid molecule is usually substantially identical to at least a portion of the target gene or genes. The nucleic acid, however, need not be perfectly identical to inhibit expression. Generally, higher homology can be used to compensate for the use of a shorter antisense nucleic acid molecule. The minimal percent identity is typically greater than about 65%, but a higher percent identity may exert a more effective repression of expression of the endogenous sequence. Substantially greater percent identity of more than about 80% typically is preferred, though about 95% to absolute identity is typically most preferred.

The antisense nucleic acid molecule need not have the same intron or exon pattern as the target gene, and non-coding segments of the target gene may be equally effective in achieving antisense suppression of target gene expression as coding segments. A DNA sequence of at least about 30 or 40 nucleotides may be used as the antisense nucleic acid molecule, although a longer sequence is preferable. In the present invention, a representative example of a useful antagonist of TSP2 is an antisense TSP2 nucleic acid molecule which is at least ninety percent identical to the complement of the TSP2 cDNA consisting of the nucleic acid sequence set forth in SEQ ID NO: 3. The nucleic acid sequence set forth in SEQ ID NO: 3 encodes the TSP2 protein consisting of the amino acid sequence set forth in SEQ ID NO: 4.

The targeting of antisense oligonucleotides to bind TSP2 mRNA is another mechanism that may be used to reduce the level of TSP2 protein synthesis. For example, the synthesis of polygalacturonase and the muscarine type 2 acetylcholine receptor are inhibited by antisense oligonucleotides directed to their respective mRNA sequences (U.S. Pat. Nos. 5,739,119 and 5,759,829). Furthermore, examples of antisense inhibition have been demonstrated with the nuclear protein cyclin, the multiple drug resistance gene (MDG1), ICAM 1, E selectin, STK-1, striatal GABA_{A} receptor and human EGF (see, e.g., U.S. Pat. Nos. 5,801,154; 5,789,573; 5,718,709 and 5,610,288).

Ribozymes can also be utilized to decrease the amount and/or biological activity of TSP2, such as ribozymes which target TSP2 mRNA. Ribozymes are catalytic RNA molecules that can cleave nucleic acid molecules having a sequence that is completely or partially homologous to the sequence of the ribozyme. It is possible to design ribozyme transgenes that encode RNA ribozymes that specifically pair with a target RNA and cleave the phosphodiester backbone at a specific location, thereby functionally inactivating the target RNA. In carrying out this cleavage, the ribozyme is not itself altered, and is thus capable of recycling and cleaving other molecules. The inclusion of ribozyme sequences within antisense RNAs confers RNA-cleaving activity upon them, thereby increasing the activity of the antisense constructs.

Ribozymes useful in the practice of the invention typically comprise a hybridizing region, of at least about nine nucleotides, which is complementary in nucleotide sequence to at least part of the target TSP2 mRNA, and a catalytic region which is adapted to cleave the target TSP2 mRNA (see generally, EPA No. 0 321 201; WO88/04300; Haseloff & Gerlach, Nature 334:585-591 [1988]; Fedor & Uhlenbeck, Proc. Natl. Acad. Sci.: USA 87:1668-1672 [1990]; Cech & Bass, Ann. Rev. Biochem. 55:599-629 [1986]).

Representative methods of delivery for antisense TSP2 molecules, and/or TSP2 ribozymes, include any of the methods of delivering nucleic acid molecules into living cells described in this patent application.

In another embodiment of this aspect of the present invention, the TSP2 antagonist is an anti-TSP2 antibody. By way of representative example, antigen useful for raising antibodies can be prepared in the following manner. A nucleic acid molecule (such as a TSP2 cDNA molecule) is cloned into a plasmid vector, such as a Bluescript plasmid (available from Stratagene, Inc., La Jolla, Calif.). The recombinant vector is then introduced into an E. coli strain (such as E. coli XL1-Blue, also available from Stratagene, Inc.) and the polypeptide encoded by the nucleic acid molecule is expressed in E. coli and then purified. Alternatively, polypeptides can be prepared using peptide synthesis methods that are well known in the art. The synthetic polypeptides can then be used to prepare antibodies. Direct peptide synthesis using solid-phase techniques (Stewart et al., Solid-Phase Peptide Synthesis, W H Freeman Co, San Francisco Calif. (1969); Merrifield, J. Am. Chem. Soc. 85:2149-2154 (1963) is an alternative to recombinant or chimeric peptide production. Automated synthesis may be achieved, for example, using Applied Biosystems 431A Peptide Synthesizer (Foster City, Calif.) in accordance with the instructions provided by the manufacturer. Methods for preparing monoclonal and polyclonal antibodies are well known to those of ordinary skill in the art and are set forth, for example, in chapters five and six of Antibodies A Laboratory Manual, E. Harlow and D. Lane, Cold Spring Harbor Laboratory (1988). Antibody production includes not only the stimulation of an immune response by injection into animals, but also analogous processes such as the production of synthetic antibodies, the screening of recombinant immunoglobulin libraries for specific-binding molecules (Orlandi et al., Proc. Natl. Acad. Sci. USA 86:3833, 1989, or Huse et al. Science 256:1275, 1989), or the in vitro stimulation of lymphocyte populations.

The invention also extends to non-antibody polypeptides, sometimes referred to as blocking peptides, that have been designed to bind specifically to, and inhibit the active site of, TSP2, or a TSP2 binding partner, or a receptor of TSP2. For example, the domain of TSP2 which binds to the receptor CD36 can be targeted with a blocking peptide. Other examples of the design of such peptides, which possess a prescribed ligand specificity are given in Beste et al. (1999, Proceedings of the National Academy of Science 96:1898-1903).

An additional strategy suitable for suppression of target gene activity entails the sense expression of a mutated or partially deleted form of the protein encoded by the target gene according to general criteria for the production of dominant negative mutations (Herskowitz I, Nature 329: 219-222 (1987)).

Representative methods of delivery for anti-TSP2 antibodies and/or blocking peptides include any of the protein delivery methods disclosed in this patent application.

In another aspect, the present invention provides medical devices comprising (a) a device body; and (b) a surface layer attached to the device body, the surface layer including an amount of an agonist or antagonist of a matricellular protein sufficient to reduce the foreign body response against the medical device, wherein the device is adapted to be affixed to, or implanted within, the soft tissue of an animal.

Some medical devices of the invention are adapted to be implanted into the soft tissue of an animal, such as a mammal, including a human, during the normal operation of the medical device. Implantable medical devices of the invention may be completely implanted into the soft tissue of an animal body (i.e., the entire device is implanted within the body), or the device may be partially implanted into an animal body (i.e., only part of the device is implanted within an animal body, the remainder of the device being located outside of the animal body). Representative examples of completely implantable medical devices include, but are not limited to: cardiovascular devices (such as vascular grafts and stents), artificial blood vessels, artificial bone joints, such as hip joints, and scaffolds that support tissue growth (in such anatomical structures as nerves, pancreas, eye and muscle). Representative examples of partially implantable medical devices include: biosensors (such as those used to monitor the level of drugs within a living body, or the level of blood glucose in a diabetic patient) and percutaneous devices (such as catheters) that penetrate the skin and link a living body to a medical device, such as a kidney dialysis machine.

Some medical devices of the invention are adapted to be affixed to soft tissue of an animal, such as a mammal, including a human, during the normal operation of the medical device. These medical devices are typically affixed to the skin of an animal body. Examples of medical devices that are adapted to be affixed to soft tissue of an animal include skin substitutes, and wound or bum treatment devices (such as surgical bandages, transdermal patches and hydrogels).

The device body can be made from any suitable material. Representative examples of synthetic polymers useful for making the device body include: (poly)urethane, (poly)carbonate, (poly)ethylene, (poly)propylene, (poly)lactic acid, (poly)galactic acid, (poly)acrylamide, (poly)methyl methacrylate and (poly)styrene. Useful natural polymers include collagen, hyaluronic acid and elastin.

The surface layer can cover the whole of the device body, or one or more parts of the device body, such as areas of the device body where it is desired to reduce the foreign body response. The surface layer can be made, for example, from any suitable material that: (a) permits deposition therein, or attachment thereto, of an amount of an agonist, or antagonist, of a matricellular protein sufficient to reduce the foreign body response against the medical device; and (b) can be attached to the device body (before or after deposition within, or attachment to, the surface layer of an amount of an agonist, or antagonist, of a matricellular protein sufficient to reduce the foreign body response against the medical device). Representative examples of materials useful for making the surface layer include porous matrices. Porous matrices are useful, for example, for delivering antisense TSP2 molecules to an animal body.

Representative porous matrices useful for making the surface layer are those prepared from tendon or dermal collagen, as may be obtained from a variety of commercial sources, (e.g., Sigma and Collagen Corporation), or collagen matrices prepared as described in U.S. Pat. Nos. 4,394,370 and 4,975,527. One collagenous material is termed UltraFiber™, and is obtainable from Norian Corp. (Mountain View, Calif.).

Certain polymeric matrices may also be employed if desired, these include acrylic ester polymers and lactic acid polymers, as disclosed, for example, in U.S. Pat. Nos. 4,526,909, and 4,563,489. Particular examples of useful polymers are those of orthoesters, anhydrides, propylene-cofumarates, or a polymer of one or more α-hydroxy carboxylic acid monomers, (e.g. α-hydroxy acetic acid (glycolic acid) and/or α-hydroxy propionic acid (lactic acid).

The surface layer can be made, for example, by attachment of matricellular protein(s) to the device body, for example by covalent activation of the surface of the medical device. By way of representative example, matricellular protein(s) can be attached to the device body by any of the following pairs of reactive groups (one member of the pair being present on the surface of the device body, and the other member of the pair being present on the matricellular protein(s): hydroxyl/carboxylic acid to yield an ester linkage; hydroxyl/anhydride to yield an ester linkage; hydroxyl/isocyanate to yield a urethane linkage.

A surface of a device body that does not possess useful reactive groups can be treated with radio-frequency discharge plasma (RFGD) etching to generate reactive groups in order to allow deposition of matricellular protein(s) (e.g., treatment with oxygen plasma to introduce oxygen-containing groups; treatment with propyl amino plasma to introduce amine groups). When an RFGD glow discharge plasma is created using an organic vapor, deposition of a polymeric overlayer occurs on the exposed surface. RFGD plasma deposited films offer several unique advantages. They are smooth, conformal, and uniform. Film thickness is easily controlled and ultrathin films (10-1000 Angstroms) are readily achieved, allowing for surface modification of a material without alteration to its bulk properties. Moreover, plasma films are highly-crosslinked and pin-hole free, and therefore chemically stable and mechanically durable. RFGD plasma deposition of organic thin films has been used in microelectronic fabrication, adhesion promotion, corrosion protection, permeation control, as well as biomaterials. (see, e.g., Ratner, U.S. Pat. No. 6,131,580).

An amount of an agonist or antagonist of a matricellular protein sufficient to reduce the foreign body response to the implanted medical device is included in or on a surface layer of the medical device. Agonists or antagonists of a matricellular protein include, for example: proteins, peptides, antibodies, and nucleic acid molecules. Useful, representative, examples of TSP2 antagonists include: TSP2 antisense nucleic acid molecules (such as antisense mRNA, antisense DNA or antisense oligonucleotides), TSP2 ribozymes, and molecules that inhibit the biological activity of TSP2 (such as anti-TSP2 antibodies, or a blocking peptide which interacts with TSP2 or a TSP2 receptor), thereby preventing TPS2 from eliciting a biological response. OPN, or OPN fragments retaining the ability to reduce the foreign body response, can be included in the surface layer of the medical device. Any combination of agonists and/or antagonists of a matricellular protein can be included in or on a surface layer of a medical device of the invention.

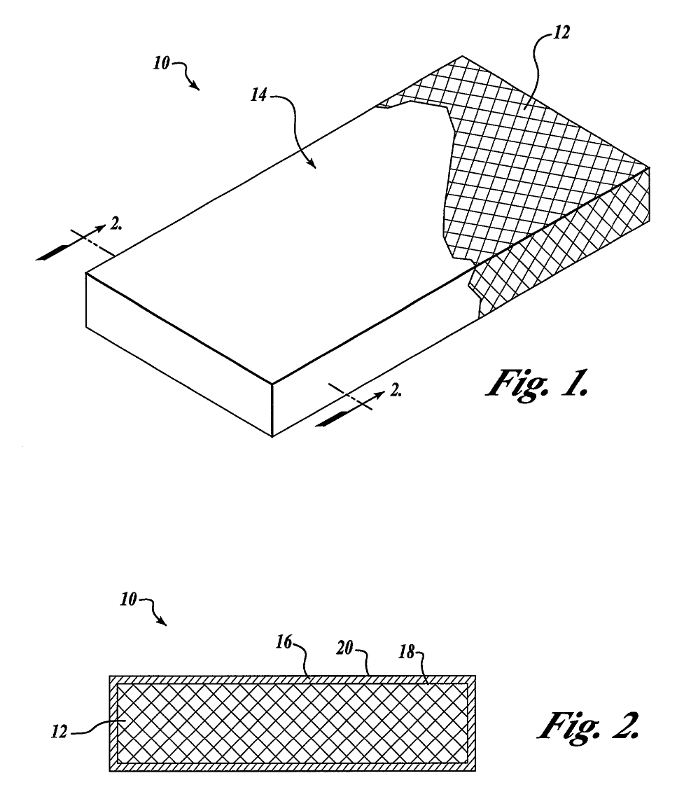

FIG. 1 shows a representative medical device 10 of the present invention, in the form of an implantable drug delivery device, which includes a device body 12 to which is attached a surface layer 14. In the embodiment shown in FIG. 1, surface layer 14 has been partially removed to show device body 12 beneath. Device body 12 is indicated by hatching. As shown in the cross-sectional view of medical device 10 in FIG. 2, surface layer 14 includes a surface layer body 16 that defines an internal surface 18, attached to device body 12, and an external surface 20.

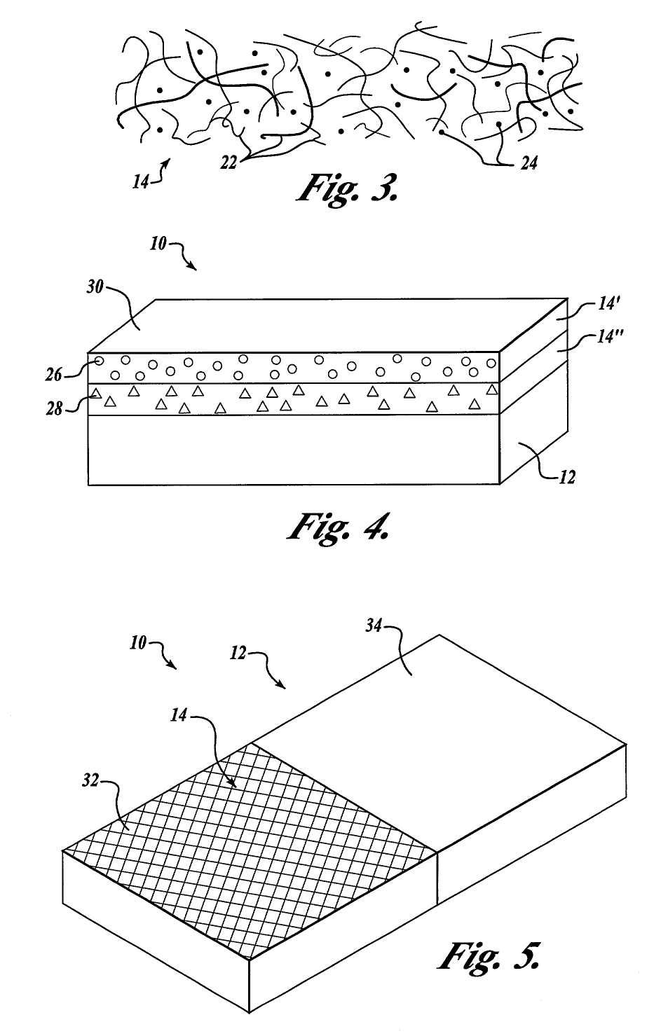

In the representative embodiment of device 10 shown in FIGS. 1 and 2, surface layer 14 is made from a porous matrix. FIG. 3 shows a representation of porous matrix 22 within which are disposed molecules 24 of an agonist or antagonist of a matricellular protein (other molecules, such as drugs, may also be disposed within porous matrix 22). Thus, in operation, device 10 is implanted into the soft tissue of an animal body where molecules 24 are released over time and reduce the foreign body response by the animal body against implanted device 10.

Some medical devices 10 of the invention include a multiplicity of surface layers 14 disposed one upon the other, wherein at least one of surface layers 14 includes an agonist or antagonist of a matricellular protein. A “multiplicity” is defined as at least two surface layers 14, and each surface layer 14 may be made from the same material as the other surface layer(s) 14, or from a different material. By way of representative example, FIG. 4 shows a medical device 10 of the invention, in the form of an implantable drug delivery device, that includes a first surface layer 14′ disposed upon a second surface layer 14″. First surface layer 14′ includes molecules of osteopontin 26 disposed therein. Second surface layer 14″ includes molecules of a thrombospondin 2 antagonist 28 disposed therein. First surface layer 14′ is located externally to second surface layer 14″ in that first surface layer 14′ is located further from device body 12 than second surface layer 14″, and first surface layer 14′ defines an external surface 30 of medical device 10. Thus, when implanted into an animal body, the embodiment of medical device 10 shown in FIG. 4 first releases osteopontin 26 into the surrounding tissue, then releases thrombospondin 2 antagonist 28 into the surrounding tissue.

FIG. 5 shows a representative embodiment of a medical device 10 of the invention, in the form of a drug delivery device, that includes a device body 12 and a surface layer 14 disposed on device body 12. Surface layer 14 includes a first area 32, including a first agonist or first antagonist of a matricellular protein, and a second area 34, including a second agonist or second antagonist of a matricellular protein. The first agonist is different from the second agonist, and the first antagonist is different from the second antagonist. Thus, for example, first area 32 can include osteopontin protein, or nucleic acid molecules encoding osteopontin, and second area 34 can include a TSP2 antagonist 28, such as a TSP2 antisense nucleic acid molecule, an immobilized anti-TSP2 antibody, or an anti-TSP2 blocking peptide.

One of ordinary skill in the art will appreciate that surface layers 14 can be configured and arranged to optimize the timing of the delivery of one or more agonists and/or antagonists of a matricellular protein in order to reduce the foreign body response. For example, typically antisense TSP2 molecules are not fixedly attached to, or within, surface layer 14 so that the antisense TSP2 molecules are free to diffuse out of surface layer 14 and be taken up by the cells of surrounding tissue. Typically, however, osteopontin protein is fixedly attached, such as by covalent linkage, to, or within, surface layer 14 to prevent movement of the protein away from the wound site. It is understood by one of ordinary skill in the art that any combination of agonist and/or antagonist of one or more matricellular proteins may be included in surface layer 14.

The following examples merely illustrate the best mode now contemplated for practicing the invention, but should not be construed to limit the invention. All literature citations herein are expressly incorporated by reference.

### EXAMPLE 1

This example describes the increase in blood vessel density that occurs within a foreign body capsule as a result of the presence of a TSP2 antisense cDNA molecule in the surface layer of an implanted device.

Construction of plasmids: Sense and antisense TSP2 expression plasmids were generated by ligation of a 3.5-kb EcoR1 fragment of mouse TSP2 (mTSP2) cDNA into the mammalian expression vector pZeoSV (Invitrogen, San Diego, Calif.). The size and orientation of inserts were confirmed by restriction digestion with Xho1.

Generation of TSP2-null mice: These mice were generated as described (Kyriakides et al., 1998, J. Cell Biol. 140: 419-430).

Preparation of devices: The devices were each made from a millipore filter coated with a collagen matrix. The collagen matrices were impregnated with a plasmid including either a TSP2 sense, or TSP2 antisense, nucleic acid molecule. Some collagen matrices were not impregnated with a plasmid. Equal amounts (1 mg) of neutralized collagen and plasmid DNA were mixed at 4° C. Implants were bathed in this solution, placed at −70° C. and then lyophilized to generate a dry gene activated matrix.

Implantation of devices: the devices were implanted into TSP2-null mice, and into control mice, for a 2-4 week period.

Measurement of capsule neovascularization: At 2-4 weeks post implant, the number of blood vessels per visual field was measured in the capsules surrounding the implant. Histological sections were stained with antibodies to PECAM-1 and visualized with the peroxidase reaction. The number and size of blood vessels was determined from microscopic digital images collected at 400× magnification and analyzed by imaging software.

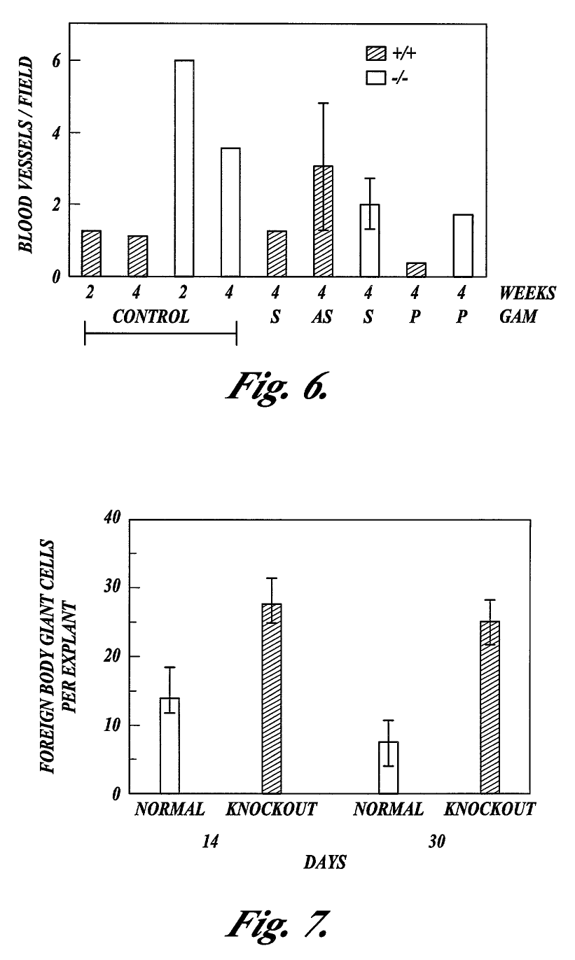

As shown in FIG. 6, foreign body capsules formed around uncoated filters implanted into TSP2-null animals displayed an increase in blood vessel density as compared to the wild type animals treated similarly. This demonstrates that in the absence of TSP2, there is an increase in neovascularization of the foreign body capsule surrounding an implant.

Under conditions designed to test TSP2 complementation, TSP2 null animals were implanted with devices comprising a millipore filter coated with a collagen matrix impregnated with a plasmid including a TSP2 cDNA in sense orientation. As shown in FIG. 6, the addition of the sense TSP2 construct led to a reduction in the vessel density within the foreign body capsule, similar to that seen in the wild-type mice, while the antisense TSP2 construct did not change the vessel density in the TSP2 null mice.

Wild-type animals implanted with a device including a surface collagen layer including an antisense TSP2 construct displayed an increase in foreign body capsule blood vessel density, while no change was observed in wild-type animals implanted with a device including a sense TSP2 construct. These results were especially significant in light of the overall reduction in vascularity observed in controls in which the collagen matrix alone was coated onto the millipore filters.

The results above suggest that in vivo delivery of TSP2 antisense cDNA via a medical device of the invention can eliminate the anti-angiogenic activity of TSP2 and thereby promote vascularization of the foreign body capsule surrounding an implanted medical device.

### EXAMPLE 2

This example shows that OPN-null mice demonstrate high levels of foreign body giant cells surrounding an implant as compared to wild type mice.

One of the hallmarks of the foreign body response is the appearance of foreign body giant cells or macrophages that have fused together as a result of encountering an implanted foreign material. As many as one hundred cells fuse to form a syncytium containing as many as one hundred nuclei. In order to address the role of OPN in the foreign body response, OPN null mice (knockout mice) and normal control mice were implanted with fixed bovine pericardium and analyzed at 14 days and 30 days post implant for the appearance of foreign body giant cells.

Generation of OPN null mice: The mice utilized in these experiments are described in Liaw, L. et al., J. Clin Invest, 1998 101(7):1468-78, which publication is incorporated herein by refernce.

Preparation of bovine pericardium implant samples: Glutaraldehyde-fixed bovine pericardial tissues were a gift from Edwards Lifesciences. Bovine pericardial tissues were excised, fixed and stored in 0.6% glutaraldehyde, pH 7.0, until use.

Method of implantation: 4 mm² biopsy punches of glutaraldehyde-fixed aortic valve leaflets (GFAV) were prepared, washed extensively in sterile PBS, and subcutaneously implanted into the dorsal side of anaesthetized 5-6 week old, female OPN +/+ or −/− mice (two GFAV per mouse). At the indicated times, mice were euthanized, and implants removed for histological analysis. All protocols were approved by the animal use committee, University of Washington.

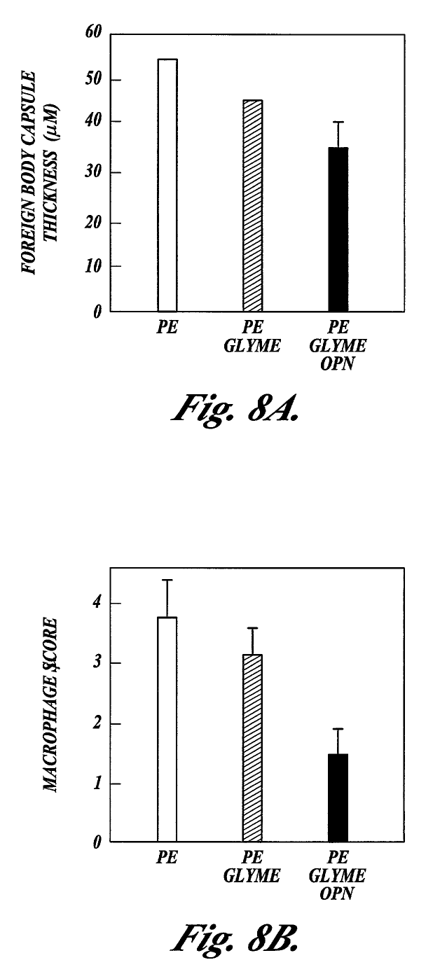

Foreign body giant cell formation: The OPN null mice and control mice were analyzed at 14 days and 30 days post implant for the appearance of foreign body giant cells. As shown in FIG. 7, at both 14 days and 30 days post-implantation, the OPN null mice had higher levels of foreign body giant cells than the control mice. These results suggest that increasing the amount and/or biological activity of OPN will decrease the number of foreign body giant cells and thereby reduce the foreign body reaction to an implant.

### EXAMPLE 3

This example shows that OPN immobilized in the surface layer of an implanted device causes a reduction in both fibrous capsule thickness and the amount of macrophage infiltration of the fibrous capsule surrounding the implanted device.

Preparation of polyethylene discs: some polyethylene discs were uncoated while others were coated with a non-fouling RFGD tetraglyme coating. Some tetraglyme-coated discs also included osteopontin that was covalently attached to the tetraglyme coating.

Tetraglyme coatings were prepared by subjecting the disks to Radio Frequency Plasma Discharge deposition of vapor phase tetra (ethylene) glycol dimethyl ether (tetra GLYME) as described in U.S. Pat. Nos. 5,153,072, and 5,002,794, (both of which patents are incorporated herein by reference). The GLYME-coated disks were sterilized with 70% ethanol/water, and filter-sterilized solutions of osteopontin were covalently immobilized to the tetraglyme coating by using disuccimidyl carbonate to activate carboxyls and hydroxyls on the glyme surface, either by reacting an allylamine glyme film with succinic anhydride, or by using the native reactive groups of the glyme film.

Method of implanting polyethylene disks: Materials were implanted in at least quadruplicate (into 4 different mice) for four weeks. Strict aseptic technique were used. All materials were sterilized by an overnight soak in sterile 70% ethanol, followed by three 20-minute washes in sterile, pyrogen free water. All instruments were autoclaved prior to surgery, and soaked in 70% ethanol between animals.

The materials were surgically implanted beneath the skin on the backs (dorsal side) of male mice using aseptic technique. Animals were anesthetized with a cocktail of ketamine and xylazine. The incision site was prepared by shaving, swabbing with Betadine followed by a 70% alcohol wipe. A single 1-1.5 cm incision was made midline on the back of each mouse, and two subcutaneous pockets were created by blunt dissection lateral to each side of the incision. One implant was placed in each pocket, and the incision was closed with sterile wound clips. Occasionally a second incision was made to accommodate two more implants, using the exact procedure as described above. Animals were allowed to recover prior to returning to housing cages. Animals were given food and water ad libidium for the remainder of the four week study.

After four weeks, animals were sacrificed by CO₂ asphyxiation, wound clips were removed, and implants were retrieved en-bloc in an effort to not disturb the biomaterial/host tissue interface. Explants were fixed with methyl Carnoy's or embedded and frozen immediately in liquid nitrogen. Chemically fixed explants were processed, embedded in paraffin and sectioned. Several sections of each explant were stained with haematoxylin and eosin (H&E) or Masson's trichrome. The remaining sections were kept in reserve for immunocytochemical staining.

Quantification of foreign body capsule thickness: Tissue samples were taken from wild-type mice at four weeks after implantation of polyethylene disks which were either uncoated, coated with a non-fouling (RFGD tetraglyme) coating, or coated with a non-fouling (RFGD tetraglyme) coating that included OPN covalently immobilized to the glyme coating. The thickness of the foreign body capsule surrounding the implants was measured. Capsule thickness was measured by light microscopy using an occular reticule that had been previously calibrated using a stage micrometer. 5 equi-distant points along the length of a single section were chosen for measurement, and capsule thickness was measured at the tissue/material interface on both surfaces of the implant (skin side and fat or muscle side) at each of these 5 points. Thus, 10 measurements were made for each implant.

As shown in FIG. 8A, the uncoated disk resulted in the thickest capsule, the glyme coating reduced the thickness of the foreign body capsule, and the glyme coating including the immobilized OPN was associated with a marked reduction in fibrous capsule thickness.

Quantification of macrophage infiltration: The tissue samples as described above were also analyzed with respect to macrophage infiltration of the foreign body capsule. As shown in FIG. 8B, the results correlated with the capsule thickness; the uncoated disks had the highest level of macrophage infiltration, followed by the glyme coating, and the lowest macrophage score was found in the glyme coating containing the immobilized OPN.

These results demonstrate that OPN immobilized on the surface of a device implanted into an animal body reduces the foreign body reaction to the implant.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method of modulating the amount or biological activity of thrombospondin 2 or osteopontin in an animal, said method comprising the step of introducing into the animal an amount of a molecule, selected from the group consisting of osteopontin and a thrombospondin 2 antagonist, effective to modulate the amount or biological activity of thrombospondin 2 or osteopontin in the animal.

2. The method of claim 1 wherein an antagonist of thrombospondin 2 is introduced into the animal.

3. The method of claim 2 wherein the amount or biological activity of thrombospondin 2 is decreased by said antagonist of thrombospondin 2.

4. The method of claim 2 wherein the thrombospondin 2 antagonist is selected from the group consisting of an antisense thrombospondin 2 nucleic acid molecule, an anti-thrombospondin 2 antibody, a thrombospondin 2 blocking peptide and a thrombospondin 2 ribozyme.

5. The method of claim 4 wherein an antisense thrombospondin 2 nucleic acid molecule is introduced into the animal.

6. The method of claim 5 wherein the antisense thrombospondin 2 nucleic acid molecule is at least ninety percent identical to the complement of a thrombospondin 2 cDNA consisting of the nucleic acid sequence set forth in SEQ ID NO. 3.

7. The method of claim 5 wherein the antisense thrombospondin 2 nucleic acid molecule hybridizes under stringent conditions to a thrombospondin 2 cDNA molecule consisting of the nucleic acid sequence set forth in SEQ ID NO. 3.

8. The method of claim 4 wherein an anti-thrombospondin 2 antibody is introduced into the animal.

9. The method of claim 4 wherein a thrombospondin 2 blocking peptide is introduced into the animal.

10. The method of claim 4 wherein a thrombospondin 2 ribozyme is introduced into the animal.

11. The method of claim 1 wherein osteopontin is introduced into the animal.

12. The method of claim 1 wherein the molecule is introduced into the animal by a method selected from the group consisting of injection, as a component of a lipid complex, as a component of an implanted porous matrix, and by immobilization onto an implanted surface.

13. The method of claim 5 wherein an antisense thrombospondin 2 nucleic acid molecule is incorporated within a delivery device which is introduced into the animal.

14. The method of claim 13 wherein the delivery device comprises a porous matrix wherein the thrombospondin 2 antisense nucleic acid molecule is disposed.

15. The method of claim 1 wherein the animal is exhibiting a wound response, and the amount of the introduced molecule is effective to [reduce] improve the wound response.

16. The method of claim 15 wherein the molecule is an antisense thrombospondin 2 nucleic acid molecule.

17. The method of claim 1 wherein osteopontin and an antagonist of thrombospondin 2 are introduced into the animal.

18. The method of claim 17 wherein the antagonist to thrombospondin 2 is an antisense thrombospondin 2 nucleic acid molecule.

19. A medical device comprising:
(a) a device body; and
(b) a surface layer attached to the device body, said surface layer comprising an amount of an agonist or antagonist of a matricellular protein sufficient to reduce the foreign body response against the device, wherein said device is adapted to be affixed to, or implanted within, the soft tissue of an animal.

20. The medical device of claim 19 wherein the device is selected from the group of devices consisting of wholly implanted medical devices, partially implanted medical devices, and surface medical devices.

21. The medical device of claim 19 wherein the surface layer attached to the device body comprises a porous matrix.

22. The medical device of claim 19 further comprising a multiplicity of surface of layers disposed one upon the other, wherein at least one of said surface layers comprises an agonist or antagonist of a matricellular protein.

23. The medical device of claim 22 wherein the device comprises: (a) a first surface layer comprising a first agonist, or first antagonist, of a matricellular protein; and (b) a second surface layer comprising a second agonist, or second antagonist, of a matricellular protein, wherein said first agonist is different from said second agonist and said first antagonist is different from said second antagonist.

24. The medical device of claim 22 wherein the device comprises: (a) a first surface layer comprising osteopontin; and (b) a second surface layer comprising a thrombospondin 2 antagonist, wherein said first surface layer is disposed external to said second surface layer.

25. The medical device of claim 24 wherein said thrombospondin 2 antagonist is an antisense nucleic acid molecule.

26. The medical device of claim 19 wherein the surface layer comprises: (a) a first area comprising a first agonist or first antagonist of a matricellular protein; and (b) a second area comprising a second agonist or second antagonist, wherein the first agonist is different from the second agonist and the first antagonist is different from the second antagonist.

27. The method of claim 1 wherein the animal is exhibiting a foreign body response, and the amount of the introduced molecule is effective to reduce the foreign body response.


SEQUENCE LISTING (4 sequences)

SEQ ID NO: 1 - DNA, 1469 bp, Homo Sapien
  CDS (102)..(1001)
agcagcaggaggaggcagagcacagcatcgtcgggaccagactcgtctcaggccagttgcagccttctcagccaaacgccgaccaaggaaaactcactaccatgagaattgcagtgatttgcttttgcctcctaggcatcacctgtgccataccagttaaacaggctgattctggaagttctgaggaaaagcagctttacaacaaatacccagatgctgtggccacatggctaaaccctgacccatctcagaagcagaatctcctagccccacagacccttccaagtaagtccaacgaaagccatgaccacatggatgatatggatgatgaagatgatgatgaccatgtggacagccaggactccattgactcgaacgactctgatgatgtagatgacactgatgattctcaccagtctgatgagtctcaccattctgatgaatctgatgaactggtcactgattttcccacggacctgccagcaaccgaagttttcactccagttgtccccacagtagacacatatgatggccgaggtgatagtgtggtttatggactgaggtcaaaatctaagaagtttcgcagacctgacatccagtaccctgatgctacagacgaggacatcacctcacacatggaaagcgaggagttgaatggtgcatacaaggccatccccgttgcccaggacctgaacgcgccttctgattgggacagccgtgggaaggacagttatgaaacgagtcagctggatgaccagagtgctgaaacccacagccacaagcagtccagattatataagcggaaagccaatgatgagagcaatgagcattccgatgtgattgatagtcaggaactttccaaagtcagccgtgaattccacagccatgaatttcacagccatgaagatatgctggttgtagaccccaaaagtaaggaagaagataaacacctgaaatttcgtatttctcatgaattagatagtgcatcttctgaggtcaattaaaaggagaaaaaatacaatttctcactttgcatttagtcaaaagaaaaaatgctttatagcaaaatgaaagagaacatgaaatgcttctttctcagtttattggttgaatgtgtatctatttgagtctggaaataactaatgtgtttgataattagtttagtttgtggcttcatggaaactccctgtaaactaaaagcttcagggttatgtctatgttcattctatagaagaaatgcaaactatcactgtattttaatatttgttattctctcatgaatagaaatttatgtagaagcaaacaaaatacttttacccacttaaaaagagaatataacattttatgtcactataatcttttgttttttaagttagtgtatattttgttgtgattatctttttgtggtgtgaataaatcttttatcttgaatgtaataagaaaaaaaaaaaaaaaacaaaaaaaaaaaa

SEQ ID NO: 2 - protein, 300 aa, Homo Sapien
MRIAVICFCLLGITCAIPVKQADSGSSEEKQLYNKYPDAVATWLNPDPSQKQNLLAPQTLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 3 - DNA, 5784 bp, Homo Sapien
  CDS (240)..(3755)
acggcatccagtacagaggggctggacttggacccctgcagcagccctgcacaggagaagcggcatataaagccgcgctgcccgggagccgctcggccacgtccaccggagcatcctgcactgcagggccggtctctcgctccagcagagcctgcgcctttctgactcggtccggaacactgaaaccagtcatcactgcatctttttggcaaaccaggagctcagctgcaggaggcaggatggtctggaggctggtcctgctggctctgtgggtgtggcccagcacgcaagctggtcaccaggacaaagacacgaccttcgaccttttcagtatcagcaacatcaaccgcaagaccattggcgccaagcagttccgcgggcccgaccccggcgtgccggcttaccgcttcgtgcgctttgactacatcccaccggtgaacgcagatgacctcagcaagatcaccaagatcatgcggcagaaggagggcttcttcctcacggcccagctcaagcaggacggcaagtccaggggcacgctgttggctctggagggccccggtctctcccagaggcagttcgagatcgtctccaacggccccgcggacacgctggatctcacctactggattgacggcacccggcatgtggtctccctggaggacgtcggcctggctgactcgcagtggaagaacgtcaccgtgcaggtggctggcgagacctacagcttgcacgtgggctgcgacctcataggaccagttgctctggacgagcccttctacgagcacctgcaggcggaaaagagccggatgtacgtggccaaaggctctgccagagagagtcacttcaggggtttgcttcagaacgtccacctagtgtttgaaaactctgtggaagatattctaagcaagaagggttgccagcaaggccagggagctgagatcaacgccatcagtgagaacacagagacgctgcgcctgggtccgcatgtcaccaccgagtacgtgggccccagctcggagaggaggcccgaggtgtgcgaacgctcgtgcgaggagctgggaaacatggtccaggagctctcggggctccacgtcctcgtgaaccagctcagcgagaacctcaagagagtgtcgaatgataaccagtttctctgggagctcattggtggccctcctaagacaaggaacatgtcagcttgctggcaggatggccggttctttgcggaaaatgaaacgtgggtggtggacagctgcaccacgtgtacctgcaagaaatttaaaaccatttgccaccaaatcacctgcccgcctgcaacctgcgccagtccatcctttgtggaaggcgaatgctgcccttcctgcctccactcggtggacggtgaggagggctggtctccgtgggcagagtggacccagtgctccgtgacgtgtggctctgggacccagcagagaggccggtcctgtgacgtcaccagcaacacctgcttggggccctcgatccagacacgggcttgcagtctgagcaagtgtgacacccgcatccggcaggacggcggctggagccactggtcaccttggtcttcatgctctgtgacctgtggagttggcaatatcacacgcatccgtctctgcaactccccagtgccccagatggggggcaagaattgcaaagggagtggccgggagaccaaagcctgccagggcgccccatgcccaatcgatggccgctggagcccctggtccccgtggtcggcctgcactgtcacctgtgccggtgggatccgggagcgcacccgggtctgcaacagccctgagcctcagtacggagggaaggcctgcgtgggggatgtgcaggagcgtcagatgtgcaacaagaggagctgccccgtggatggctgtttatccaacccctgcttcccgggagcccagtgcagcagcttccccgatgggtcctggtcatgcggcttctgccctgtgggcttcttgggcaatggcacccactgtgaggacctggacgagtgtgccctggtccccgacatctgcttctccaccagcaaggtgcctcgctgtgtcaacactcagcctggcttccactgcctgccctgcccgccccgatacagagggaaccagcccgtcggggtcggcctggaagcagccaagacggaaaagcaagtgtgtgagcccgaaaacccatgcaaggacaagacacacaactgccacaagcacgcggagtgcatctacctgggtcacttcagcgaccccatgtacaagtgcgagtgccagacaggctacgcgggcgacgggctcatctgcggggaggactcggacctggacggctggcccaacctcaatctggtctgcgccaccaacgccacctaccactgcatcaaggataactgcccccatctgccaaattctgggcaggaagactttgacaaggacgggattggcgatgcctgtgatgatgacgatgacaatgacggtgtgaccgatgagaaggacaactgccagctcctcttcaatccccgccaggctgactatgacaaggatgaggttggggaccgctgtgacaactgcccttacgtgcacaaccctgcccagatcgacacagacaacaatggagagggtgacgcctgctccgtggacattgatggggacgatgtcttcaatgaacgagacaattgtccctacgtctacaacactgaccagagggacacggatggtgacggtgtgggggatcactgtgacaactgccccctggtgcacaaccctgaccagaccgacgtggacaatgaccttgttggggaccagtgtgacaacaacgaggacatagatgacgacggccaccagaacaaccaggacaactgcccctacatctccaacgccaaccaggctgaccatgacagagacggccagggcgacgcctgtgaccctgatgatgacaacgatggcgtccccgatgacagggacaactgccggcttgtgttcaacccagaccaggaggacttggacggtgatggacggggtgatatttgtaaagatgattttgacaatgacaacatcccagatattgatgatgtgtgtcctgaaaacaatgccatcagtgagacagacttcaggaacttccagatggtccccttggatcccaaagggaccacccaaattgatcccaactgggtcattcgccatcaaggcaaggagctggttcagacagccaactcggaccccggcatcgctgtaggttttgacgagtttgggtctgtggacttcagtggcacattctacgtaaacactgaccgggacgacgactatgctggcttcgtctttggttaccagtcaagcagccgcttctatgtggtgatgtggaagcaggtgacgcagacctactgggaggaccagcccacgcgggcctatggctactccggcgtgtccctcaaggtggtgaactccaccacggggacgggcgagcacctgaggaacgcgctgtggcacacggggaacacgccggggcaggtgcgaaccttatggcacgaccccaggaacattggctggaaggactacacggcctataggtggcacctgactcacaggcccaagaccggctacatcagagtcttagtgcatgaaggaaaacaggtcatggcagactcaggacctatctatgaccaaacctacgctggcgggcggctgggtctatttgtcttctctcaagaaatggtctatttctcagacctcaagtacgaatgcagagatatttaaacaagatttgctgcatttccggcaatgccctgtgcatgccatggtccctagacacctcagttcattgtggtccttgcggcttctctctctagcagcacctcctgtcccttgaccttaactctgatggttcttcacctcctgccagcaaccccaaacccaagtgccttcagaggataaatatcaatggaactcagagatgaacatctaacccactagaggaaaccagtttggtgatatatgagactttatgtggagtgaaaattgggcatgccattacattgctttttcttgtttgtttaaaaagaatgacgtttacatataaaatgtaattacttattgtatttatgtgtatatggagttgaagggaatactgtgcataagccattatgataaattaagcatgaaaaatattgctgaactacttttggtgcttaaagttgtcactattcttgaattagagttgctctacaatgacacacaaatcccgctaaataaattataaacaagggtcaattcaaatttgaagtaatgttttagtaaggagagattagaagacaacaggcatagcaaatgacataagctaccgattaactaatcggaacatgtaaaacagttacaaaaataaacgaactctcctcttgtcctacaatgaaagccctcatgtgcagtagagatgcagtttcatcaaagaacaaacatccttgcaaatgggtgtgacgcggttccagatgtggatttggcaaaacctcatttaagtaaaaggttagcagagcaaagtgcggtgctttagctgctgcttgtgccgttgtggcgtcggggaggctcctgcctgagcttccttccccagctttgctgcctgagaggaaccagagcagacgcacaggccggaaaaggcgcatctaacgcgtatctaggctttggtaactgcggacaagttgcttttacctgatttgatgatacatttcattaaggttccagttataaatattttgttaatatttattaagtgactatagaatgcaactccatttaccagtaacttattttaaatatgcctagtaacacatatgtagtataatttctagaaacaaacatctaataagtatataatcctgtgaaaatatgaggcttgataatattaggttgtcacgatgaagcatgctagaagctgtaacagaatacatagagaataatgaggagtttatgatggaaccttaatatataatgttgccagcgattttagttcaatatttgttactgttatctatctgctgtatatggaattcttttaattcaaacgctgaaaacgaatcagcatttagtcttgccaggcacacccaataatcagtcatgtgtaatatgcacaagtttgtttttgtttttgttttttttgttggttggtttttttgctttaagttgcatgatctttctgcaggaaatagtcactcatcccactccacataaggggtttagtaagagaagtctgtctgtctgatgatggatagggggcaaatctttttcccctttctgttaatagtcatcacatttctatgccaaacaggaacgatccataactttagtcttaatgtacacattgcattttgataaaattaattttgttgtttcctttgaggttgatcgttgtgttgttttgctgcactttttacttttttgcgtgtggagctgtattcccgagacaacgaagcgttgggatacttcattaaatgtagcgactgtcaacagcgtgcaggttttctgtttctgtgttgtggggtcaaccgtacaatggtgtgggaatgacgatgatgtgaatatttagaatgtaccatattttttgtaaattatttatgtttttctaaacaaatttatcgtataggttgatgaaacgtcatgtgttttgccaaagactgtaaatatttatttatgtgttcacatggtcaaaatttcaccactgaaaccctgcacttagctagaacctcatttttaaagattaacaacaggaaataaattgtaaaaaaggttttct

SEQ ID NO: 4 - protein, 1172 aa, Homo Sapien
MVWRLVLLALWVWPSTQAGHQDKDTTFDLFSISNINRKTIGAKQFRGPDPGVPAYRFVRFDYIPPVNADDLSKITKIMRQKEGFFLTAQLKQDGKSRGTLLALEGPGLSQRQFEIVSNGPADTLDLTYWIDGTRHVVSLEDVGLADSQWKNVTVQVAGETYSLHVGCDLIGPVALDEPFYEHLQAEKSRMYVAKGSARESHFRGLLQNVHLVFENSVEDILSKKGCQQGQGAEINAISENTETLRLGPHVTTEYVGPSSERRPEVCERSCEELGNMVQELSGLHVLVNQLSENLKRVSNDNQFLWELIGGPPKTRNMSACWQDGRFFAENETWVVDSCTTCTCKKFKTICHQITCPPATCASPSFVEGECCPSCLHSVDGEEGWSPWAEWTQCSVTCGSGTQQRGRSCDVTSNTCLGPSIQTRACSLSKCDTRIRQDGGWSHWSPWSSCSVTCGVGNITRIRLCNSPVPQMGGKNCKGSGRETKACQGAPCPIDGRWSPWSPWSACTVTCAGGIRERTRVCNSPEPQYGGKACVGDVQERQMCNKRSCPVDGCLSNPCFPGAQCSSFPDGSWSCGFCPVGFLGNGTHCEDLDECALVPDICFSTSKVPRCVNTQPGFHCLPCPPRYRGNQPVGVGLEAAKTEKQVCEPENPCKDKTHNCHKHAECIYLGHFSDPMYKCECQTGYAGDGLICGEDSDLDGWPNLNLVCATNATYHCIKDNCPHLPNSGQEDFDKDGIGDACDDDDDNDGVTDEKDNCQLLFNPRQADYDKDEVGDRCDNCPYVHNPAQIDTDNNGEGDACSVDIDGDDVFNERDNCPYVYNTDQRDTDGDGVGDHCDNCPLVHNPDQTDVDNDLVGDQCDNNEDIDDDGHQNNQDNCPYISNANQADHDRDGQGDACDPDDDNDGVPDDRDNCRLVFNPDQEDLDGDGRGDICKDDFDNDNIPDIDDVCPENNAISETDFRNFQMVPLDPKGTTQIDPNWVIRHQGKELVQTANSDPGIAVGFDEFGSVDFSGTFYVNTDRDDDYAGFVFGYQSSSRFYVVMWKQVTQTYWEDQPTRAYGYSGVSLKVVNSTTGTGEHLRNALWHTGNTPGQVRTLWHDPRNIGWKDYTAYRWHLTHRPKTGYIRVLVHEGKQVMADSGPIYDQTYAGGRLGLFVFSQEMVYFSDLKYECRDI